(19) United States
(12) Patent Application Publication
(10) Pub. No.: US 2024/0382256 A1
(43) Pub. Date: Nov. 21, 2024

(54) METHOD OF INTRODUCING A BARE FIBER TIP INTO A HANDPIECE, SCOPE, OR CANNULA WITHOUT DAMAGE

(71) Applicant: OPTICAL INTEGRITY, INC., PANAMA CITY BEACH, FL (US)

(72) Inventors: JOE D. BROWN, PANAMA CITY BEACH,, FL (US); DANIEL MALPHURS, PANAMA CITY BEACH,, FL (US)

(21) Appl. No.: 18/664,684

(22) Filed: May 15, 2024

Related U.S. Application Data
(60) Provisional application No. 63/502,262, filed on May 15, 2023.

(51) Int. Cl. A61B 18/22; A61B 17/00; A61B 90/92

(52) U.S. Cl. CPC A61B 18/22; A61B 90/92; A61B 2017/00526; A61B 2018/2244

(57) ABSTRACT

A method of preparing a laser delivery fiber for a therapeutic, treatment, or surgical procedure includes steps of providing an optical fiber having a pre-formed tip; placing a temporary protective sleeve over the pre-formed tip; inserting the optical fiber with the protective sleeve through a handpiece; and, before beginning the therapeutic, treatment, or surgical procedure, removing the protective sleeve from the optical fiber.

This application claims the benefit of Provisional Appl. Ser. No. 63/502,262, filed May 15, 2023, and incorporated herein by reference.

### BACKGROUND OF THE INVENTION

### 1. Field of the Invention

The invention relates to the field of laser therapy, treatment, or surgery, and in particular to a method of preparing for a surgical laser procedure.

Therapeutic, treatment, or surgical procedures to which the method of the invention may be applied include ear, nose, and throat (ENT) procedures, as well as any other surgical procedure that utilizes a handpiece, scope, or cannula, into which a bare fiber is to be inserted before the fiber is used in the procedure. By “bare” fiber is meant an optical fiber from which buffer layers have been removed, leaving only the fiber core and cladding, and which is introduced to the patent and utilized during surgery without an additional sleeve or ferrule surrounding the tip of the fiber.

The method of the invention includes the steps of placing a temporary protective sleeve over the bare fiber tip, inserting the fiber through the handpiece, and removing the temporary protective sleeve from the bare fiber tip before use of the optical fiber during a therapeutic, treatment, or surgical procedure.

The method of the invention enables use of pre-stripped and pre-formed fiber tips, without risk of damage to the fiber tip or handpiece as the fiber is inserted therethrough.

### 1. DESCRIPTION OF RELATED ART

For certain procedures that utilize a handpiece, scope, or cannula to guide or position an optical fiber during the procedure, it is common to complete preparation of the optical fiber for surgery at the hospital or clinic in which the procedure is to be performed, by having the surgeon or a surgical assistant or technician carry out stripping of the fiber and cleaving of the glass fiber core, rather than utilizing a factory finished fiber tip. This involves having the surgeon or assistant pass a non-stripped fiber through the handpiece, scope, or cannula (hereinafter referred-to as a “handpiece”), followed by stripping of the fiber buffer from the core, and manual cleaving of the glass tip to form a planar end surface. The reason that the fiber is inserted through the handpiece before stripping and cleaving of the fiber tip is that a stripped and cleaved fiber tip is more vulnerable to damage as it is inserted through the scope, and also more likely to damage the inside surface of the handpiece.

Manually stripping and cleaving the optical fiber after insertion into the handpiece avoids the possibility of damage to the finished fiber tip, and/or the inside surface of the handpiece, but has several disadvantages: First, manual cleaving of the glass fiber core by the surgeon or assistant lacks the precision of a machine-formed fiber tip, prepared at a fiber production facility or factory. A manually cleaved fiber tip typically has a laser output pattern that is nonsymmetrical, in comparison with the precision output patterns achievable by optical fiber tips prepared at a facility with dedicated tip-forming equipment, including by way of example and not limitation, angled, conical, rounded, ball, or lensed tips. Second, the manual strippers used during on-site preparation of the optical fiber, following insertion of the optical fiber through the handpiece, have not yet been validated for auto-clave sterilization. As a result, auto-clave cannot legally be used to sterilize the fiber tip stripping tool, necessitating less convenient and more costly sterilization procedures, or subjecting the hospital or clinic to penalties for violating FDA rules.

A need therefore exists for a way to allow a finished optical fiber tip to be inserted through a handpiece without risk of damaging the optical fiber or the handpiece, thereby avoiding the need to wait to strip and cleave the fiber tip until after the fiber is inserted through the handpiece, and enabling or facilitating use of pre-stripped and formed fiber tips.

One solution to the problem of preventing damage to a bare fiber and/or handpiece before a therapeutic, treatment, or surgical procedure was proposed in the inventor's U.S. Patent Publication No. 2017/0042618, which disclosed disposable or single-use sleeves configured to be placed over the tip of the fiber to protect the fiber tip assembly from contamination or debris-build up during the procedure, and also during insertion into a handpiece. However, because the previously-proposed protective sleeve was intended to remain on the fiber during treatment, or at least until the fiber was at a treatment position within the patient, the protective sleeve had to be made out of surgical-grade materials approved for insertion into the patient. Such sleeves add to the cost of the treatment procedure and may pose some risk to the patient if detached from the fiber during the procedure.

A variety of additional protective sleeves, including sleeves that serve as a stand-off to prevent contact between a laser tip and a target of the laser, have also been disclosed in the inventor's U.S. Pat. No. 11,278,352. All of these sleeves can serve to protect the fiber tip during insertion through a scope, but do not address the problem of preparing a fiber for procedures that require a bare fiber tip inserted through a handpiece, and that do not utilize a sleeve during the procedure.

### SUMMARY OF THE INVENTION

It is accordingly a first objective of the invention to provide a method of preparing an optical fiber for a therapeutic or surgical procedure without risk of damage to either the optical fiber or the handpiece as the optical fiber is inserted into the handpiece, and without requiring that the fiber tip to be stripped and cleaved only after insertion through the handpiece has been completed.

It is a second objective of the invention to provide a method of preparing a bare optical fiber, i.e., one that does not use a protective sleeve or ferrule configured to remain on the fiber during a therapeutic or surgical procedure, without having to strip and cleave the bare optical fiber only after the bare optical fiber has been inserted through the handpiece.

It is a further objective of the invention to provide a method of preparing a laser delivery fiber for a surgical laser procedure that permits use of pre-formed fiber tips, without risk of damage to a handpiece through which the pre-formed fiber tips are inserted.

These objectives are achieved by a method of preparing a fiber for a laser surgery procedure that includes the steps of:
providing an optical fiber having a pre-formed tip;
placing a temporary protective sleeve over the pre-formed tip;
inserting the optical fiber with the protective sleeve through the handpiece; and
before beginning a surgical procedure and/or introducing the optical fiber into a patient, removing the protective sleeve from the fiber.

This objective is further achieved, in accordance with the principles of a preferred embodiment of the invention, by a method of preparing a fiber for a laser surgery procedure that includes the steps of:
providing a surgical laser delivery fiber having a pre-formed tip and an intact (unstripped) buffer;
stripping a section of the buffer from the fiber tip;
placing the stripped section of buffer over the tip of the fiber, and/or moving the stripped section of buffer back toward the unstripped position, before inserting the fiber into the handpiece;
inserting the fiber, together with the stripped section of the buffer, through the handpiece; and
removing the stripped section of buffer from the tip of the fiber after insertion of the fiber through the handpiece, at which time a treatment procedure may be carried out.

Although the invention uses a temporary protective sleeve that is removed from the optical fiber after insertion of the optical fiber through the handpiece and before the optical fiber is introduced into a patient during a therapeutic or surgical procedure, in contrast to the disposable, single use, and/or standoff sleeves disclosed for example in the inventor's U.S. Patent Publication No. 2017/0042618 and U.S. Pat. No. 11,278,352, it will be appreciated that the present invention is not limited to a particular type or configuration of the protective sleeve. Any protective sleeve, including sleeves that resemble those disclosed in U.S. Patent Publication No. 2017/0042618 and U.S. Pat. No. 11,278,352, as well as sleeve that utilize different materials and have different shapes, may be used to protect the fiber during insertion into and through the handpiece, so long as the protective sleeve can be removed before introduction into a patent and commencement of a treatment procedure.

Therapeutic, treatment, or surgical procedures to which the method of the invention may be applied include any surgical procedure that utilizes a handpiece, scope, or cannula, into which a bare fiber is to be inserted before the fiber is introduced into a patient and used in the procedure. The procedures may include, but are not limited to, ear, nose, and throat (ENT) procedures

### BRIEF DESCRIPTION OF THE DRAWINGS

FIG. 1 is a flowchart of a method for preparing for a therapeutic, treatment, or surgical laser procedure according to the principles of a preferred embodiment of the current invention.

FIG. 2 is an isometric view of an optical fiber with a pre-formed tip and a section of stripped buffer, for use in the method of FIG. 1.

FIG. 3 is an isometric view illustrating a step of placing a temporary protective sleeve over the tip of the fiber.

FIG. 4 is an isometric view of an optical fiber with a preformed tip and a temporary protective sleeve, during insertion of the optical fiber through a handpiece.

FIG. 5 is a perspective view of the optical fiber with the preformed tip, after insertion through the handpiece and removal of the protective sleeve.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

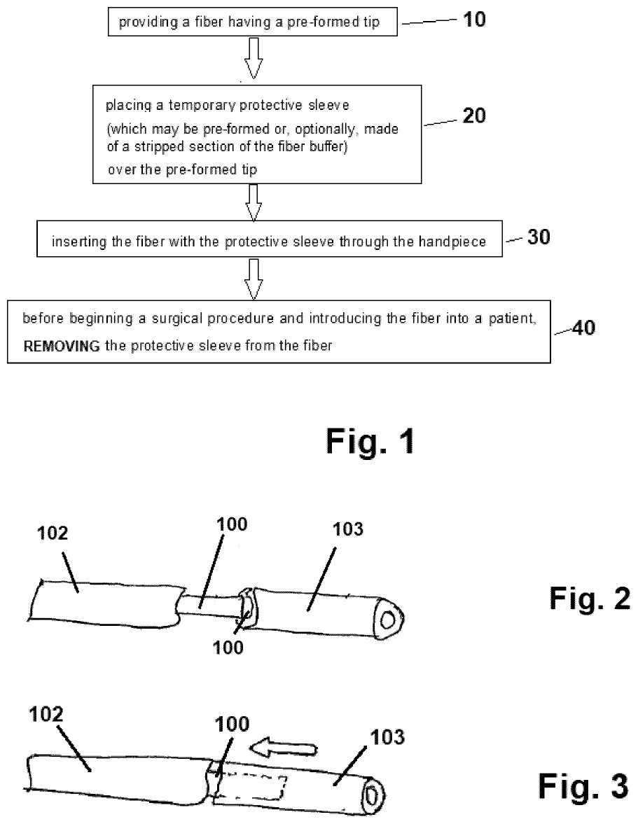

As shown in FIG. 1, the method of the invention includes the following steps: a step 10 of providing an optical fiber 100 having a pre-formed tip 101;
a step 20 of placing a temporary protective sleeve 103 over the pre-formed tip;
a step 30 of inserting the optical fiber with the pre-formed tip and temporary protective sleeve 103 through the handpiece 104; and
a step 40 of, before introducing the optical fiber into a patient and/or beginning
a surgical procedure, removing the protective sleeve 103 from the optical fiber 100.

The pre-formed tip 101 may have a cone shape, a ball shape, a flat surface oriented either transversely or at another nonzero angle with respect to an axis of the optical fiber 100, or any other shape, depending on the desired direction and shape of the beam as it exits the fiber. Optical fibers with appropriately shaped tips are commercially available and the present invention is not limited to a particular optical fiber type or tip configuration.

Since the temporary protective sleeve 103 is not inserted into the patient and not used during a treatment procedure, it may be made of any material capable of being sterilized and fitted over the end of fiber tip 101, so long as the temporary protective sleeve 103 is capable of being removed from the fiber following insertion of the optical fiber through the handpiece 104. Preferably, the material is sufficiently resilient to, or includes structures that, grip and hold the temporary protective sleeve on the bare optical fiber core or cladding during insertion through the handpiece, while still allowing removal by the technician after insertion through the handpiece has been completed. Alternatively, the protective sleeve may be adhered to the stripped optical fiber by a temporary adhesive such as the pressure sensitive acrylate (PSA) used in Post-It™ notes, or by a mechanical fastener. Suitable materials for the protective sleeve include rubber or a polymer such as PTFE or ETFE, although any material with suitable protective and fiber-gripping properties may be utilized.

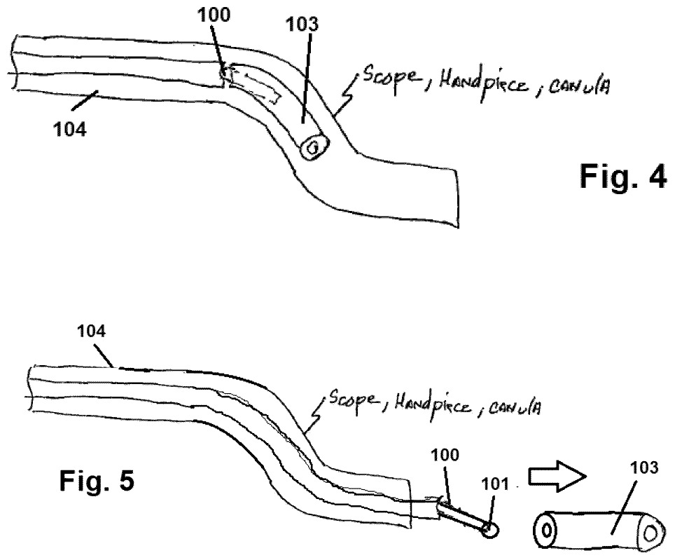

In the embodiment illustrated in FIGS. 2-5, the temporary protective sleeve 103 is made of a section of the fiber buffer 102 that has been stripped from the optical fiber 100, or from another optical fiber, and that is placed back over the fiber tip 101 during insertion into the handpiece 104. The use of a section of fiber buffer as a “standoff” sleeve during surgery is known from U.S. Pat. No. 11,278,352, but in the method of the present invention, the sleeve 103 is removed from the optical fiber 100 following insertion through the handpiece but before surgery begins.

Because the temporary protective sleeve 103 used in the method of the invention is not subjected to heat or bodily fluids, but rather is removed before any treatment procedure begins, it can be reused multiple times. To facilitate stripping it is possible, but not necessary, to use a different color for the end section of the fiber buffer. The fiber itself is not limited to a particular type of laser or procedure, and can have either an end-firing or side-firing tip configuration.

What is claimed is:

1. A method of preparing for a therapeutic or surgical procedure, comprising steps of:
providing an optical fiber having a pre-formed tip;
placing a temporary protective sleeve over the pre-formed tip;
inserting the optical fiber with the protective sleeve through the handpiece; and
before beginning the therapeutic or surgical procedure, removing the protective sleeve from the therapeutic or surgical fiber.

2. A method of preparing for a therapeutic or surgical procedure as claimed in claim 1, wherein the temporary protective sleeve is configured to grip the therapeutic or surgical fiber during insertion through the handpiece, while enabling removal of the temporary protective sleeve from the optical fiber by a technician after completion of insertion through the handpiece.

3. A method of preparing for a therapeutic or surgical procedure as claimed in claim 1, wherein the temporary protective sleeve is made of PTFE or ETFE.

4. A method preparing for a therapeutic or surgical procedure as claimed in claim 1, wherein the temporary protective sleeve is a section of a buffer of the optical fiber that has been stripped from the optical fiber or from another optical fiber.

5. A method preparing for a therapeutic or surgical procedure as claimed in claim 4, wherein the temporary protective sleeve has a color that is different than a color of the buffer of the optical fiber.

6. A method preparing for a therapeutic or surgical procedure as claimed in claim 1, wherein the temporary protective sleeve is reusable.
